(19) United States
(12) Patent Application Publication
(10) Pub. No.: US 2019/0159974 A1
(43) Pub. Date: May 30, 2019

(54) NAIL COMPOSITION SET CONTAINING MIRROR PIGMENT

(71) Applicant: L'ORÉAL, Paris (FR)

(72) Inventors: Sam Morrison, Clark, NJ (US); Charles Ward, Burlington, NJ (US); Ramakrishnan Hariharan, Springfield, NJ (US); Xavier Blin, Westfield, NJ (US)

(73) Assignee: L'ORÉAL, Paris (FR)

(21) Appl. No.: 15/827,743

(22) Filed: Nov. 30, 2017

(51) Int. Cl. A61K 8/03; A61Q 3/02; A61K 8/02; A61K 8/26; A61K 8/34

(52) U.S. Cl. CPC A61K 8/03; A61Q 3/02; A61K 8/0254; A61K 8/26; A61K 2800/884; A61K 2800/26; A61K 2800/43; A61K 2800/436; A61K 8/34

(57) ABSTRACT

The invention relates to nail composition sets including mirror pigment, as well as to methods and kits related to such composition sets.

### FIELD OF THE INVENTION

The present invention relates to nail composition sets comprising a basecoat composition comprising mirror pigment and a topcoat composition comprising polar solvent and at least one film-forming agent, as well as to methods of maintaining mirror properties and improving wear properties of a basecoat composition which has been applied to a nail comprising mirror pigment by applying a topcoat composition comprising polar solvent and at least one film-forming agent to a basecoat composition comprising mirror pigment.

### DISCUSSION OF THE BACKGROUND

Mirror pigments are known. For example, Eckart markets mirror pigments under the brand name of Lunaris® and/or Silverdream®.

Further, use of mirror pigments or similar pigments in nail basecoat compositions is known.

U.S. Pat. No. 6,565,835, US patent application 2008/0131383, PCT patent application WO 02/03913, U.S. Pat. No. 7,507,285, PCT patent application WO 07/014680 may be relevant.

However, nail basecoat compositions containing mirror pigments have poor wear and shine properties. And it is difficult to maintain mirror properties after the basecoat composition has been applied.

There remains a need for new ways to maintain mirror properties and improve wear properties of nail basecoat compositions comprising mirror pigment.

### SUMMARY OF THE INVENTION

The present invention relates to a nail composition set comprising a basecoat composition comprising mirror pigment and a topcoat composition comprising polar solvent and at least one film-forming agent. Preferably, the topcoat composition comprises at least one film-forming agent which comprises styrene.

The present invention also relates to a kit comprising at least one basecoat composition comprising mirror pigment and at least one topcoat composition comprising polar solvent and at least one film-forming agent. Preferably, the topcoat composition comprises at least one film-forming agent which comprises styrene.

The present invention also relates to methods of maintaining mirror properties and/or improving wear properties of a basecoat composition which has been applied to a nail comprising mirror pigment comprising applying a topcoat composition comprising polar solvent and at least one film-forming agent to the basecoat composition. Preferably, the topcoat composition comprises at least one film-forming agent which comprises styrene.

The present invention further relates to methods for making up and/or protecting nails comprising applying to the nails a nail basecoat composition comprising mirror pigment comprising applying a topcoat composition comprising polar solvent and at least one film-forming agent to the basecoat composition. Preferably, the topcoat composition comprises at least one film-forming agent which comprises styrene.

It is to be understood that both the foregoing general description and the following detailed description are exemplary and explanatory only, and are not restrictive of the invention.

### DETAILED DESCRIPTION OF THE INVENTION

As used herein, the expression “a” and “at least one” mean one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

“Film-former” or “film-forming agent,” as used herein means a polymer or resin that leaves a film on the substrate to which it is applied, for example, after a solvent accompanying the film former has evaporated, absorbed into and/or dissipated on the substrate.

“Adhesion,” as used herein, refers to chemical or physical bonding between a coating and a substrate. Good adhesion between nail polish and nail surface should translate to good wear properties on consumers. Adhesion properties can be quantified by in-vitro method such as a cross-cut adhesion test. In the test, a lattice pattern is cut into the coating and penetrates through to the substrate. A pressure sensitive tape is applied to the sample and then pulled off. The adhesion property can be quantified by the area of the coating remaining after peeling. For example, if the whole film remains after peeling, it indicates excellent adhesion. If most of the film gets peeled off, it indicates poor adhesion. The cross-cut test is an industrial standard test for testing adhesion for coatings. (Reference # ISO/DIN 2409, ASTM D3359).

“Substituted,” as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Nail” as used herein includes fingernails as well as toenails.

“Volatile”, as used herein, means having a flash point of less than about 100° C.

“Non-volatile”, as used herein, means having a flash point of greater than about 100° C.

“Shine” or “gloss,” as used herein, refers to surface shininess. Gloss meters are commonly used in the nail composition art, and can measure the amount of light reflected from the surface or film of interest. Gloss may be quantified, for example, as a % reflectance at 20° or at 60° angles. Shine/gloss can be determined according to the following procedure: films are drawn down onto Laneta Form 5C—Opacity cards and allowed to dry. Gloss measurements can be made using a BYK micro-TRI-gloss with results reported at the reflectance angle at which measurements were taken (for example, at 20° or at 60°) and expressed by gloss units (GU). Preferably, basecoat compositions of the present invention have shine properties of from 300 to 500 GU at 20° or at 60°, preferably from 350 to 475 GU at 20° or at 60°, and preferably from 400 to 450 GU at 20° or at 60°, including all ranges and subranges therebetween.

“Haze,” as used herein, refers to a measurement of dispersion in glossy surfaces. It is often manifested as “milkiness” or “cloudiness.” Haze may be quantified, for example, as a % reflectance at 20° or at 60° angles. Haze can be determined according to the following procedure: films are drawn down onto Laneta Form 5C—Opacity cards and allowed to dry. Haze measurements can be made using a BYK micro-haze plus meter with results reported at the reflectance angle at which measurements were taken (for example, 20° or at 60°) and expressed by haze units (HU). Preferably, topcoat compositions of the present invention have haze properties of from 575 to 750 HU at 20°, preferably from 600 to 700 HU at 20°, and preferably from 625 to 675 HU at 20°, including all ranges and subranges therebetween. Preferably, the topcoat compositions have haze properties of 700 HU or less.

“Mirror properties” or “mirror effect,” as used herein, refers to reflection of light to produce a visual effect. Reflection of light (reflectivity) can be determined quantitatively, for example, by determining the ratio of incident light upon a surface to the amount of light reflected from the surface. “Mirror properties” or “mirror effect” can be quantified, for example, by using techniques for determining shine or gloss such as those described in the preceding paragraph.

The compositions, sets and methods of the present invention can comprise, consist of, or consist essentially of the essential elements and limitations of the invention described herein, as well as any additional or optional ingredients, components, or limitations described herein or otherwise useful. For example, the film-forming component of the topcoat composition can “consist essentially of” or “consist of” film-forming agent comprising styrene.

In this regard, the “film-forming component” of the topcoat composition comprises film-forming agent comprising styrene and optionally may contain one or more additional film-forming agents. Preferably, the film-forming component contains a majority (greater than 50% by weight) of film-forming agent comprising styrene, preferably greater than 60% by weight, preferably greater than 70% by weight, preferably greater than 80% by weight, preferably greater than 90% by weight, and may contain up to 100% by weight of film-forming agent comprising styrene, all percentages being based on weight of film-forming agent comprising styrene with respect to the total weight of the film-forming component. All ranges and subranges within the percentages set forth above are included herein such as, for example, 10%-100% by weight, 25%-90% by weight, 50%-85% by weight, etc.

For purposes of the topcoat compositions and components of the present invention which “consist essentially of” identified ingredients, the “basic and novel property” of such compositions and components is “maintaining mirror properties of a basecoat composition which has been applied to a nail comprising mirror pigment.”

For purposes of the methods of the present invention directed to maintaining mirror properties and improving wear properties of a basecoat composition which has been applied to a nail comprising mirror pigments by applying topcoat compositions which “consist essentially of” identified ingredients, the “basic and novel propert(ies)” of such compositions is the property associated with the identified purpose(s) of the method. So, for example, “maintaining mirror properties of a basecoat composition which has been applied to a nail comprising mirror pigment” is the basic and novel property associated with methods of maintaining mirror properties of a basecoat composition which has been applied to a nail comprising mirror pigment.

Basecoat Composition

According to the present invention, a basecoat composition comprising mirror pigment is provided.

A mirror pigment is a coloring agent which has mirror properties. Preferably, the mirror pigment of the present invention is leafed or platelet-shaped having different length and width characteristics.

Preferred length to thickness ratios of the mirror pigments are 5 to 250, preferably 7 to 200, preferably 8 to 150, and preferably 10 to 100, including all ranges and subranges therebetween.

Preferred width to thickness ratios of the mirror pigments are 5 to 250, preferably 7 to 200, preferably 8 to 150, and preferably 10 to 100, including all ranges and subranges therebetween.

Preferred thicknesses of the mirror pigments are 1 to 250 microns, preferably 3 to 175 microns, and preferably 5 to 100 microns, including all ranges and subranges therebetween.

Preferably, the mirror pigment of the present invention is a metal-based pigment, most preferably an aluminum pigment. Also preferably, the mirror pigment of the present invention comprises a surface treatment which is hydrophobic and/or oleophobic. Preferably, the surface treatment is hydrophobic.

According to preferred embodiments, the surface treatment comprises at least one fatty acid. “Fatty acid” refers to a long aliphatic chain carboxylic acid compound, either saturated or unsaturated and either branched or unbranched. Preferably, the fatty acid is saturated and unbranched. The fatty acid preferably comprises an aliphatic chain comprising from 4 to 36 carbon atoms, preferably from 8 to 32 carbon atoms, preferably from 14 to 28 carbon atoms, and preferably from 16 to 22 carbon atoms, including all ranges and subranges therebetween. Suitable examples include, but are not limited to, stearic acid, isostearic acid, oleic acid, palmitic acid, and behenic acid.

Examples of suitable mirror pigments are marketed by Eckart under the brand name of Lunaris® and/or Silverdream®. For example, a suitable mirror pigment has the INCI designation of STYRENE/METHYLSTYRENE COPOLYMER (and) ALUMINUM POWDER.

Preferably, the mirror pigment is present in the basecoat compositions of the present invention in amounts ranging from about 0.05% to about 5%, preferably from about 0.075% to about 1%, and preferably from about 0.1% to about 0.5%, by weight, based on the total weight of the composition, including all ranges and subranges in between.

Topcoat Composition

According to the present invention, a topcoat composition comprising polar solvent and at least one film-forming agent is provided.

Polar Solvent

According to the present invention, topcoat compositions comprising at least one polar solvent are provided. According to preferred embodiments, the topcoat compositions of the present invention comprise at least one polar solvent selected from the group consisting of water, C2-C5 alcohols, and mixtures thereof. Suitable C2-C5 alcohols include ethanol, propanol, butanol, pentanol, isopropanol, isobutanol and isopentanol.

Preferably, the polar solvent(s) is/are present in the topcoat compositions of the present invention in amounts ranging from about 0.5% to about 50%, preferably from about 1% to about 40%, and preferably from about 5% to about 25%, by weight, based on the total weight of the composition, including all ranges and subranges in between.

Preferably, topcoat compositions of the present invention comprise from about 5% to about 50% water, preferably from about 10% to about 45% water, preferably from about 15% to about 40% water, and preferably from about 20% to about 40% water by weight with respect to the total weight of the composition, including all ranges and subranges therebetween.

Preferably, the solvent component of the topcoat compositions of the present invention consists essentially of, or consists of, water, C2-C5 alcohols, optionally plasticizing agent, and mixtures thereof. That is, the topcoat composition does not contain any solvent in an amount which has a material adverse effect on the mirror properties of the basecoat composition.

Preferably, the compositions of the present invention are substantially free of non-polar solvents (i.e., contain less than about 1% non-polar solvents). In other embodiments, the compositions are devoid of non-polar solvents (i.e., contain less than about 0.1% non-polar solvents).

Film-Forming Agent

According to the present invention, topcoat compositions comprising at least one film-forming agent are provided. According to preferred embodiments, the topcoat compositions of the present invention comprise at least one dispersion of film forming particles in aqueous phase. The dispersion of film forming particles in aqueous phase is more generally known as latex.

Preferably, the polymers for the film-forming particles may be selected from vinyl (co)polymers, (meth)acrylic (co)polymers, urethanes (co)polymers, salts (such as ammonium) thereof, and mixtures thereof. Advantageously, the polymer for the film-forming particles is selected from a styrene-(meth)acrylic and (meth)acrylic copolymer, a vinyl acetate and (meth)acrylic copolymer, salts (such as ammonium) and mixtures thereof.

Polymers for the film-forming particles of the free-radical type may be chosen, for example, from vinyl polymers or copolymers, such as acrylic polymers.

Vinyl film-forming polymers can result from the polymerization of monomers comprising at least one ethylenic unsaturation and at least one acidic group and/or esters of these acidic monomers and/or amides of these acidic monomers. Monomers comprising at least one acid group which may be used include, for example, α,β-ethylenic unsaturated carboxylic acids such as acrylic acid, methacrylic acid, crotonic acid, maleic acid or itaconic acid. (Meth)acrylic acid and crotonic acid are, for example, used. Preferably, (meth)acrylic acid is used.

The esters of acidic monomers can be chosen, for example, from (meth)acrylic acid esters (also known as (meth)acrylates), such as (meth)acrylates of an alkyl, for example, a C1-C30 alkyl, such as a C1-C20 alkyl, (meth)acrylates of an aryl, such as a C6-C10 aryl, and (meth)acrylates of a hydroxyalkyl, such as a C2-C6 hydroxyalkyl. Among the alkyl (meth)acrylates that may be mentioned, examples include methyl methacrylate, ethyl methacrylate, butyl methacrylate, isobutyl methacrylate, 2-ethylhexyl methacrylate, lauryl methacrylate and cyclohexyl methacrylate. Among the hydroxyalkyl (meth)acrylates that may be mentioned, examples include hydroxyethyl acrylate, 2-hydroxypropyl acrylate, hydroxyethyl methacrylate and 2-hydroxypropyl methacrylate. Among the aryl (meth)acrylates that may be mentioned, examples include benzyl acrylate and phenyl acrylate. The (meth)acrylic acid esters that may be used are, for example, alkyl (meth)acrylates.

The alkyl group of the esters may be substituted. For example, the alkyl group of the esters may be either fluorinated or perfluorinated, i.e., some or all of the hydrogen atoms of the alkyl group are substituted with fluorine atoms. Further, examples of amides of the acid monomers that may be mentioned include (meth)acrylamides, such as N-alkyl(meth)acrylamides, for example, of a C₂-C₁₂ alkyl. Among the N-alkyl(meth)acrylamides that may be mentioned, examples include N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For example, these monomers may be polymerized with acid monomers and/or esters thereof and/or amides thereof, such as those mentioned above. Examples of vinyl esters that may be mentioned include vinyl acetate, vinyl neodecanoate, vinyl pivalate, vinyl benzoate and vinyl t-butylbenzoate. Styrene monomers that may be mentioned include styrene and α-methylstyrene.

Among the film-forming polycondensates that may be mentioned, examples include polyurethanes, polyesters, polyesteramides, polyamides, epoxyester resins and polyureas, and modifications or derivatives of any of these.

The polyurethanes may be chosen from anionic, cationic, nonionic or amphoteric polyurethanes, polyurethane-acrylics, polyurethane-polyvinylpyrrolidones, polyester-polyurethanes, polyether-polyurethanes, polyureas and polyurea-polyurethanes, and mixtures thereof.

The polyesters may be obtained, in a known manner, by polycondensation of dicarboxylic acids with polyols, such as diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalene-dicarboxylic acid and 2,6-naphthalenedicarboxylic acid. These dicarboxylic acid monomers may be used alone or as a combination of at least two dicarboxylic acid monomers. Among these monomers, phthalic acid, isophthalic acid and terephthalic acid may, for example, be used.

The diol may be chosen from aliphatic, alicyclic and aromatic diols. The diol used is, for example, chosen from ethylene glycol, diethylene glycol, triethylene glycol, 1,3-propanediol, cyclohexanedimethanol and 4-butanediol. Other polyols that may be used include glycerol, pentaerythritol, sorbitol and trimethylolpropane.

The polyesteramides may be obtained in a manner analogous to that of the polyesters, by polycondensation of diacids with diamines or amino alcohols. Diamines that may be used include, for example, ethylenediamine, hexamethylenediamine and meta- or para-phenylenediamine. An amino alcohol that may be used is, for example, monoethanolamine.

The polyester may also comprise at least one monomer bearing at least one —SO₃M group, wherein M is chosen from a hydrogen atom, an ammonium ion NH₄⁺ and a metal ion such as an Na⁺, Li⁺, K⁺, Mg²⁺, Ca²⁺, Cu²⁺, Fe²⁺ or Fe³⁺ ion. A difunctional aromatic monomer comprising such an —SO₃M group may, for example, be used.

The aromatic nucleus of the difunctional aromatic monomer also comprising an —SO₃M group as described above may be chosen, for example, from benzene, naphthalene, anthracene, biphenyl, oxybiphenyl, sulfonylbiphenyl and methylenebiphenyl nuclei. Among the difunctional aromatic monomers also comprising an —SO₃M group, mention may be made, for example, of sulfoisophthalic acid, sulfoterephthalic acid, sulfophthalic acid, 4-sulfonaphthalene-2,7-dicarboxylic acid.

The copolymers used are, for example, those based on isophthalate/sulfoisophthalate, such as copolymers obtained by condensation of diethylene glycol, cyclohexanedimethanol, isophthalic acid and sulfoisophthalic acid.

The polymer for the film forming particles may also be a liposoluble polymer. Examples of the liposoluble polymer that may be mentioned include copolymers of a vinyl ester (wherein the vinyl group is directly linked to the oxygen atom of the ester group and the vinyl ester comprises a radical chosen from saturated, linear or branched hydrocarbon-based radicals of 1 to 19 carbon atoms, linked to the carbonyl of the ester group) and of at least one other monomer, which may be a vinyl ester (different from the vinyl ester already present), an α-olefin (comprising from 8 to 28 carbon atoms), an alkyl vinyl ether (the alkyl group of which comprises from 2 to 18 carbon atoms) or an allylic or methallylic ester (comprising a radical chosen from saturated, linear or branched hydrocarbon-based radicals of 1 to 19 carbon atoms, linked to the carbonyl of the ester group).

These copolymers may be crosslinked using crosslinking agents that may be either of the vinylic type or of the allylic or methallylic type, such as tetraallyloxyethane, divinylbenzene, divinyl octanedioate, divinyl dodecanedioate and divinyl octadecanedioate.

Examples of these copolymers which may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Further examples of the liposoluble film-forming polymers include liposoluble copolymers, such as those resulting from the copolymerization of vinyl esters comprising from 9 to 22 carbon atoms or of alkyl acrylates or methacrylates, wherein the alkyl radicals comprise from 10 to 20 carbon atoms. Such liposoluble copolymers may be chosen, for example, from polyvinyl stearate, polyvinyl stearate crosslinked with the aid of divinylbenzene, of diallyl ether or of diallyl phthalate copolymers, polystearyl (meth)acrylate, polyvinyl laurate and polylauryl (meth)acrylate copolymers, it being possible for these poly(meth)acrylates to be crosslinked with the aid of ethylene glycol dimethacrylate or tetraethylene glycol dimethacrylate. The liposoluble copolymers described above are known and are described, for example, in French patent application FR-A-2 232 303; they may have a weight-average molecular weight ranging, for example, from 2,000 to 500,000 such as from 4,000 to 200,000.

Among the liposoluble film-forming polymers which may be used herein, mention may also be made, for example, of polyalkylenes such as copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) such as copolymers of vinylpyrrolidone and of C₂-C₄₀ alkene such as C₃-C₂₀ alkene. Among the VP copolymers which may be used herein, mention may be made, for example, of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Specific examples of aqueous dispersions of film-forming particles which may be used are the acrylic dispersions sold under the names “Neocryl XK-90®”, “Neocryl A-1070®”, “Neocryl A-1090®”, “Neocryl BT-62®”, “Neocryl A-1079®” and “Neocryl A-523®” by the company Avecia-Neoresins, “Dow Latex 432®” by the company Dow Chemical, “Daitosol 5000 AD®” or “Daitosol 5000 SJ” by the company Daito Kasey Kogyo; the aqueous dispersions of polyurethane sold under the names “Neorez R-981®” and “Neorez R-974®” by the company Avecia-Neoresins, “Avalure UR-405®”, “Avalure UR-410®”, “Avalure UR-425®”, “Avalure UR-450®”, “Sancure 875®”, “Sancure 861R®”, “Sancure 878®” and “Sancure 2060®” by the company Goodrich, “Impranil 85®” by the company Bayer and “Aquamere H-151®” by the company Hydromer; vinyl dispersions, for instance “Mexomer PAM” and also acrylic dispersions in isododecane, for instance “Mexomer PAP” by the company Chimex.

Further specific examples of latex polymers for use in the present invention further include ammonium acrylates copolymer, ethylhexyl acrylate/hema copolymer (and) acrylates/diethylaminoethyl methacrylate/ethylhexyl acrylate copolymer (Syntran®PC 5775), styrene/acrylates/ammonium methacrylate copolymer (Syntran®5760, Syntrar®5009, Syntran®PC5620), polyacrylate-21 (and) acrylates/dimethylaminoethyl methacrylate copolymer (Syntran®PC5100, Syntran®PC5776, Eudragit®E 100, Jurymer ET-410C), styrene/acrylates/ammonium methacrylate copolymer (Syntran®5009 CG), olefin/acrylate grafted polymer (and) sodium laureth sulfate (and C12-15 SEC-pareth 15 (Syntran®DEX108), acrylates copolymer (Aculyn®33A Polymer, Avalure®Ace 210/120/315 Acrylic Copolymer, Carbopol® Aqua SF-1 Polymer, Coatex®Co 633, Eliclear®380/70014U, Eudragit® L 100, Joncryl®85, Luviflex®Soft), acrylates/ethylhexyl acrylate copolymer. The Syntran® polymers are commercially available from the supplier Interpolymer Corp.

According to preferred embodiments of the present invention, nail compositions comprising at least one film-forming agent comprising styrene are provided. Suitable examples include, but are not limited to, styrene/acrylic/ammonium methacrylic copolymers, styrene/acrylic copolymers, modified styrene/acrylic/ammonium methacrylic copolymers, modified styrene/acrylic copolymers, and mixtures thereof. It should be understood that, as used above, “(meth)acrylic” refers both to (meth)acrylic and (meth)acrylate.

According to preferred embodiments, the at least one film-forming agent comprising styrene contains more than 50% styrene by weight, preferably 60% or more styrene by weight, preferably 70% or more styrene by weight, and preferably 80% or more styrene by weight, with respect to the total weight of the film forming agent. Representative examples of specific film forming agents comprising styrene include, but are not limited to, acrylic copolymer dispersions sold under the names Neocryl XK-90® (INCI name: acrylic/styrene copolymer), Neocryl A-1070® (INCI name: acrylic/styrene copolymer), Neocryl A-1090® (INCI name: acrylic/styrene copolymer), Neocryl BT-62® (INCI name: acrylic/styrene copolymer), Neocryl A-1079® (INCI name: acrylic/styrene copolymer), Neocryl A-523® (INCI name: acrylic/styrene copolymer) by the company Avecia-Neoresins, Dow Latex 432® (INCI name: Styrene/Acrylates Copolymer) by the company Dow Chemical, Neocryl A-2091 (INCI name: Modified Styrene Acrylic Copolymer Dispersion) SYNTRAN™ 5760 (styrene/acrylates/ammonium methacrylate copolymer (and) butylene glycol (and) sodium laureth-12 sulphate (INCI name), commercially available from Interpolymer Corporation); JONCRYL™ 77 (styrene/acrylates copolymer in the form of an ammonia salt, along with water and polypropylene glycol, available from BASF Performance Chemicals); and RHOPLEX™ P376 (styrene/acrylates copolymer available from Dow Chemical Company).

Preferably, the film-forming agents(s) is/are present in the topcoat compositions of the present invention in amounts of active material (e.g., solid content) ranging from about 0.5% to about 50%, preferably from about 1% to about 40%, and preferably from about 5% to about 35%, by weight, based on the total weight of the composition, including all ranges and subranges in between.

Preferably, the polar solvent(s) and film-forming agent(s) are present in the topcoat compositions of the present invention in weight ratios of polar solvent(s) to film-forming agent(s) (solid content) of from about 10:1 to about 1:3, preferably about 5:1 to about 1:2 and preferably about 3:1 to about 1:1, including all ranges and subranges therebetween.

Other Ingredients

According to the present invention, the basecoat and/or topcoat composition may comprise at least one other ingredient typically found in nail compositions. One of ordinary skill in the art would readily understand the types of ingredients typically found in nail compositions. A non-exhaustive list of such ingredients includes, but is not limited to, cellulose compounds, film-forming agents, plasticizers, coalescing agents, and non-mirror coloring agents.

Suitable cellulose compounds include, but are not limited to, cellulose polymers, such as, for example, hydroxyethylcellulose, hydroxypropylcellulose, methylcellulose, ethylhydroxyethylcellulose and nitrocellulose.

According to preferred embodiments, the cellulose compound is nitrocellulose. Particularly preferred nitrocellulose compounds include nitrocellulose resins having a nitrogen content from 10% to 12.5% by weight, preferably 10.5% to 11.5% by weight, and preferably from 10.7% to 11.3% by weight with respect to the weight of the nitrocellulose compound, including all ranges and subranges therebetween. Preferred nitrocellulose compounds are soluble in C1-C4 alcohols, preferably ethanol (C2).

According to preferred embodiments, the at least one cellulose compound, if present, is present in the compositions of the present invention in an amount of active material ranging from about 0.01 to about 30% by weight, more preferably from about 0.1 to about 20% by weight, and most preferably from about 1 to about 10% by weight, based on the total weight of the composition, including all ranges and subranges within these ranges.

The plasticizing component of the compositions of the present invention may optionally contain one or more plasticizers.

Plasticizers (plasticizing agents) are additives used to optimize the mechanical properties of the films. They tend to reduce the Glass Transition Temperature (Tg) and increase the softness and flexibility of the films. Preferably, the plasticizer has a distribution coefficient D of less than or equal to 0.1. The distribution coefficient can be determined in accordance with the teaching of “A method to predict the distribution coefficient of coalescing agents between latex particles and the water phase,” Progress in Organic Coatings, vol. 30, 1997, pp. 173-177, the disclosure of which is specifically incorporated by reference herein.

Preferably, the plasticizer has a boiling point measured at ambient pressure of less than or equal to 285° C., preferably less than or equal to 270° C., and preferably less than or equal to 250° C. In the present specification, the boiling point values are to be considered accurate to ±2° C. owing to the uncertainties of boiling point measurement.

Any plasticizing agent typically found in nail polish compositions can be used. Examples of suitable plasticizers include, but are not limited to, glycols and their ester derivatives, esters of acids, in particular carboxylic acids, such as citrates, adipates, carbonates, tartrates, phosphates or sebacates, oxyethylenated derivatives, such as oxyethylenated oils, and their mixtures. For example, suitable plasticizing agents include, but are not limited to, diisobutyl adipate, the ester of teributyl acid and 2,2,4-trimethylpentane-1,3-diol, diethyl adipate, diethyl phthalate, dibutyl phthalate, dioctyl phthalate, butyl 2-ethylhexyl phthalate, dimethyl sebacate, dibutyl sebacate, ethyl stearate, 2-ethylhexyl palmitate, dipropylene glycol n-butyl ether, tributyl phosphate, tributoxyethyl phosphate, tricresyl phosphate, triphenyl phosphate, glycerol triacetate, butyl stearate, butyl glycolate, butyl acetyltricinoleate, glyceryl acetyltricinoleate, dibutyl phthalate, diisobutyl phthalate, dioctyl phthalate, dimethoxyethyl phthalate, diamyl phthalate, triethyl citrate, tributyl citrate, tributyl acetylcitrate, tri(2-ethylhexyl) acetylcitrate, dibutyl tartrate, camphor, and mixtures thereof.

In accordance with preferred embodiments, the plasticizing component is present in the compositions of the present invention in an amount of from 2.5% to 50% by weight, preferably from 4% to 40% by weight, preferably from 5% to 30% by weight, of the total weight of the composition, including all ranges and subranges therebetween.

According to preferred embodiments, the compositions of the present invention are substantially free of phthalates (i.e., contain less than about 0.5% phthalates). In other embodiments, the compositions are free of phthalates (i.e., contain less than about 0.25% phthalates) or devoid of phthalates (i.e., contain less than about 0.1% phthalates).

The film-forming component of the topcoat compositions of the present invention may optionally contain one or more film-formers other than the latex film-forming agents discussed above. The basecoat compositions of the present invention may optionally contain one or more film-forming agents, including the latex film forming agents discussed above.

Suitable film forming agents include water-soluble film forming agents and oil-soluble film forming agents.

Specific examples of suitable water-soluble film forming agents include, but are not limited to, latexes, proteins, such as proteins of plant origin, such as, for example, wheat or soya proteins; or proteins of animal origin, such as keratins, for example keratin hydrolysates and sulfonic keratins; acrylic polymers or copolymers, such as, for example, polyacrylates or polymethacrylates; vinyl polymers, such as, for example, polyvinylpyrrolidones, copolymers of methyl vinyl ether and of maleic anhydride, the copolymer of vinyl acetate and of crotonic acid, copolymers of vinylpyrrolidone and of vinyl acetate, copolymers of vinylpyrrolidone and of caprolactam, or polyvinyl alcohol; gums arabic, guar gum, xanthan derivatives or karaya gum; alginates and carrageenans; glycoaminoglycans, hyaluronic acid and its derivatives; shellac resin, gum sandarac, dammars, elemis or copals; muccopolysaccharides, such as chondroitin sulfates; and their mixtures.

In accordance with preferred embodiments, the film-forming component is present in the compositions of the present invention in an amount of from 0.5% to 50% by weight, preferably from 1% to 40% by weight, preferably from 5% to 30% by weight, of the total weight of the composition, including all ranges and subranges therebetween.

Coalescents (coalescing agents) are additives used assist the film formation process of certain film forming agents (e.g., latex). Preferably, the coalescent agent has a distribution coefficient D′ of greater than or equal to 0.5, measured in accordance with the above-referenced “A method to predict the distribution coefficient of coalescing agents between latex particles and the water phase,” Progress in Organic Coatings, vol. 30, 1997, pp. 173-177. Preferably, the coalescent agent has a boiling point measured at ambient pressure ranging from 90° C. to 180° C., preferably from 150° C. to 180° C.

Any coalescent agent typically found in nail polish compositions can be used. Examples of suitable plasticizers include, but are not limited to, propylene glycol n-butyl ether, dipropylene glycol dimethyl ether, propylene glycol methyl ether acetate, propylene glycol propyl ether, methyl lactate, ethyl lactate, isopropyl lactate, and mixtures thereof.

In accordance with preferred embodiments, the coalescent agent, if present, is present in the compositions of the present invention in an amount of from 0.1% to 25% by weight, preferably from 1% to 15% by weight, preferably from 3 to 10% by weight, of the total weight of the composition, including all ranges and subranges therebetween.

Suitable non-mirror colorants (non-mirror coloring agents) include any colorant typically found in nail compositions. Suitable non-mirror colorants include, but are not limited to, lipophilic dyes, pigments, and their mixtures.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium oxides, cerium oxides, tin oxides, iron oxides, or chromium oxides, manganese violet, ultramarine blue, chromium hydrate and ferric blue. Mention may also be made, among organic pigments, of carbon black, pigments of D & C type and lakes based on carmine or on barium, strontium, calcium or aluminum, such aluminum powder, barium sulfate, D&C Red No. 10, 11, 12, and 13, D&C Red No. 7, D&C Red No. 5 and 6, and D&D Red No. 34, as well as lakes such as D&C Blue Lake No. 2, D&C Yellow Lake No. 5 and D&C Red Lake No. 2.

In accordance with preferred embodiments, the non-mirror colorant, if present, is preferably present in an amount sufficient to provide color to the nail, preferably in an amount of from about 0.1% to about 50% by weight, preferably from about 0.25% to about 25% by weight, preferably from about 0.33% to about 10% by weight and preferably from about 0.5 to about 1% by weight, of the total weight of the composition, including all ranges and subranges therebetween.

Suitable pearlesecent pigments can be chosen from pigments having a substrate (for example, mica or synthetic fluorophlogopite) which are coated with metal oxide (for example, oxide of titanium, iron, chromium, zirconium, silicon and/or tin) and pigments having a metallic substrate (for example, aluminum) which are coated with an oxide (for example, silica) and optionally may contain additional coatings (for example, a metallic layer such as silver). Preferably, the particle size of the pearlescent pigment is from 1 to 150 microns, preferably 3 to 100 microns, and preferably 5 to 50 microns, including all ranges and subranges therebetween.

For example, suitable pearlescent pigments can be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, pearlescent pigments based on bismuth oxychloride, and aluminum flake covered by silica as an interferential layer and coated with silver particle as an outer layer such as aluminum powder (and) silica (and) silver (and) tin oxide.

Specific suitable pearlescent pigments include, for example:

Sunshine spectral golden, TITANIUM DIOXIDE AND SYNTHETIC FLUORPHLOGOPITE AND IRON OXIDE (PARTICLE SIZE: 5-35 μM), INCI Name: SYNTHETIC FLUORPHLOGOPITE (and) TITANIUM DIOXIDE (and) IRON OXIDES (and) TIN OXIDE;

Visionaire honey, BRONZE POWDER (ALLOY OF COPPER 85% AND ZINC 10%) COATED WITH SILICA;

Intenza passionate kiss SYNTHETIC FLUORPHLOGOPITE AND TITANIUM DIOXIDE RED 7 CALCIUM LAKE ON BARIUM SULFATE SUBTRACT (SIZE 5-35 μM);

Soft sparkle sunflower, WHITE MICA PEARL COATED WITH TITANIUM DIOXIDE (10-100 MICRONS), INCI Name: TITANIUM DIOXIDE (and) MICA (and) SILICA (and) TIN OXIDE;

Kiwi rose, SILICA, TITANIUM DIOXIDE, & TIN OXIDE (54/43/3) (PARTICLE SIZE 5-50 μM);

Syncrystal scarlet, MICA SYNTHETIC (FLUORPHLOGOPITE) AND TITANIUM DIOXIDE AND TIN OXIDE AND CARMINE (CI:77891) (SIZE 10-50 μM);

Celeste rose pink, ALUMINUM FLAKE COVERED BY SILICA AS INTERFRENTIAL LAYER AND COATED WITH SILVER PARTICLE AS OUTER LAYER (54/22/23), INCI Name: ALUMINUM POWDER (and) SILVER (and) SILICA (and) TIN OXIDE; and

Cosmicolor Iris blue, ALUMINUM FLAKE COVERED BY SILICA AS INTERFRENTIAL LAYER AND COATED WITH SILVER PARTICLE AS OUTER LAYER (65/25/9), INCI Name: ALUMINUM POWDER (and) SILICA (and) SILVER (and) TIN OXIDE.

In accordance with preferred embodiments, the pearlescent pigments, if present, are preferably present in an amount of from about 0.1% to about 1.5% by weight, preferably from about 0.25% to about 1.25% by weight, preferably from about 0.33% to about 1% by weight and preferably from about 0.5 to about 1% by weight, of the total weight of the composition, including all ranges and subranges therebetween.

In accordance with preferred embodiments, pearlescent pigments are present in the basecoat compositions of the present invention in a pearlescent pigment to mirror pigment weight ratio of about 20:1 to about 1:1, preferably about 5:1 to about 1:1, and preferably about 3:1 to about 1.5:1, including all ranges and subranges therebetween.

Nail Composition Set

According to the present invention, nail composition sets comprising (1) at least one basecoat comprising mirror pigment and (2) at least one topcoat comprising polar solvent and at least one film-forming agent are provided. Preferably, the topcoat composition comprises at least one film-forming agent which comprises styrene.

For example, a nail composition set comprising at least one basecoat, at least one primer and at least one topcoat are provided. However, the primer is optional.

It should be understood that each coat or layer in the nail composition set, itself, can comprise one or more layers of each composition. Thus, the at least one basecoat can comprise one or more basecoat layers; and the at least one topcoat can comprise one or more topcoat layers. Preferably, each basecoat and topcoat contains three or fewer layers or compositions, more preferably two or fewer layers or compositions, and most preferably a single layer or composition.

Generally speaking, the basecoat composition and topcoat composition which can be applied to form the nail composition set can be any suitable solvent-based composition for application to nails. Examples of suitable conventional solvent-based compositions can be found, for example, in U.S. Pat. Nos. 7,455,831, 7,025,953, 6,555,096, 6,372,201, 6,333,025, and 6,254,878, the entire contents of all of which are hereby incorporated by reference in their entireties.

During application of the nail composition set, the primer (if used) is applied to the nail. The basecoat is applied to the primer (if used); if primer is not used, the basecoat is applied to the nail. Then, the topcoat is applied to the basecoat. In this manner, a nail composition set comprising a primer (optional), a basecoat and a topcoat can be prepared on a nail.

Auxiliaries/Additives

The compositions discussed above may additionally comprise an additive or auxiliary commonly used in cosmetic compositions and known to a person skilled in the art as being capable of being incorporated into a nail polish or varnish composition. Such additives or auxiliaries may be chosen from solvents, thickeners, coalescents, preservatives, fragrances, oils, waxes, surfactants, antioxidants, agents for combating free radicals, spreading agents, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, fibers, and the like, and their mixtures.

A person skilled in the art will take care to select the optional additional additives and/or the amount thereof such that the advantageous properties of the compositions according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the compositions of the invention should be cosmetically or dermatologically acceptable, i.e., they should contain a non-toxic physiologically acceptable. The compositions may be in any galenic form normally employed in the cosmetic and dermatological fields which is suitable for topical administration onto nails.

According to some embodiments, compositions of the invention may further comprise at least one organic solvent. Suitable examples of solvents, include, but are not limited to, ketones which are liquid at room temperature such as, for example, methyl ethyl ketone, methyl isobutyl ketone, diisobutyl ketone, isophorone, cyclohexanone, and acetone; glycols which are liquid at room temperature, such as ethylene glycol, propylene glycol, pentylene glycol, and glycerol; propylene glycol ethers which are liquid at room temperature, such as propylene glycol monomethyl ether, propylene glycol monomethyl ether acetate, and dipropylene glycol mono-n-butyl ether; short-chain esters (having from 3 to 8 carbon atoms in total) such as ethyl acetate, methyl acetate, propyl acetate, n-butyl acetate, and isopentyl acetate; ethers which are liquid at room temperature, such as diethyl ether, dimethyl ether, and dichlorodiethyl ether; alkanes which are liquid at room temperature such as decane, heptane, dodecane, isododecane, and cyclohexane; aromatic cyclic compounds which are liquid at room temperature, such as toluene and xylene; and aldehydes which are liquid at room temperature, such as benzaldehyde and acetaldehyde.

If present, the organic solvent preferably comprises from about 10% to about 95% by weight, preferably from about 30% to about 90% by weight, and preferably from about 50% to about 85% by weight, relative to the total weight of the composition.

However, the compositions of the present invention may also lack any and all such organic solvents, and may lack in particular ethyl acetate, methyl acetate, propyl acetate, n-butyl acetate, and/or isopentyl acetate.

According to preferred embodiments of the present invention, methods for making up and/or protecting nails comprising applying to the nails (1) at least one basecoat comprising mirror pigment; and (2) at least one topcoat comprising polar solvent and at least one film-forming agent are provided. Preferably, the topcoat composition comprises at least one film-forming agent which comprises styrene.

“Making up” as used herein means to provide decoration (for example, color) to the nail. “Protecting” as used herein means to inhibit damage to the nail (for example, chipping) by providing a protective layer on the nail.

In accordance with preferred embodiments of the preceding methods, at least one basecoat and at least one topcoat are applied topically to the nails of a person in need of (desirous) the desired making up or protection in an amount sufficient to achieve the desired result. The coats may be applied to the desired area as needed.

According to preferred embodiments of the present invention, a kit comprising (1) at least one basecoat composition comprising mirror pigment; and (2) at least one topcoat comprising polar solvent and at least one film-forming agent are provided. Preferably, the topcoat composition comprises at least one film-forming agent which comprises styrene.

The compositions according to the invention can be manufactured by known processes used generally in the cosmetics or dermatological field.

According to preferred embodiments of the present invention, methods of maintaining mirror properties and/or improving wear properties of a basecoat composition which has been applied to a nail comprising mirror pigment comprising applying a topcoat composition comprising polar solvent and at least one film-forming agent to the basecoat composition are provided. Preferably, the topcoat composition comprises at least one film-forming agent which comprises styrene.

Preferably, in the methods of maintaining mirror properties of the basecoat composition which has been applied to a nail discussed herein, “maintaining mirror properties” results in at least 80% of the original mirror properties remaining two hours after application of the topcoat to the basecoat, preferably at least 85% and preferably at least 90% the mirror properties.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

The following basecoat and topcoat compositions were prepared:

Basecoat Composition

| Ingredient | Concentration |
|---|---|
| Alkyl Acetates | 58 |
| Styrene/methylstyrene copolymer | 39 |
| Alcohol | 2.7 |
| Aluminum Powder | 0.3 |
| Total | 100 |

Topcoat Composition

| Ingredient | Concentration |
|---|---|
| SIMETHICONE | 0.000476 |
| DIVINYLDIMETHICONE/DIMETHICONE | 0.3 |
| COPOLYMER | |
| SODIUM LAURETH SULFATE | 0.08700 |
| STYRENE/ACRYLATES/AMMONIUM | 35.49791 |
| METHACRYLATE | |
| COPOLYMER | |
| PROPYLENE GLYCOL BUTYL ETHER | 0.114285 |
| DISODIUM LAURETH SULFOSUCCINATE | 0.00228 |
| AMMONIUM ACRYLATES COPOLYMER | 0.274296 |
| SORBIC ACID | 0.00028 |
| CAPRYLYL GLYCOL | 0.26672 |
| PHENOXYETHANOL | 0.73990 |
| BHT | 0.00018 |
| PROPYLENE GLYCOL BUTYL ETHER | 2.17123 |
| DIMETHICONE | 0.00476 |
| BENZOIC ACID | 0.000238 |
| C12-13 PARETH-3 | 0.0105 |
| C12-13 PARETH-23 | 0.0105 |
| SODIUM LAURYL SULFATE | 0.08929 |
| SULFURIC ACID | 0.00004 |
| WATER | 56.23766 |
| METHYLCELLULOSE | 0.000476 |
| POLYSORBATE 65 | 0.001428 |
| DIPROPYLENE GLYCOL DIBENZOATE | 4.1905 |

The mirror properties of the nail set containing the basecoat and topcoat were determined as follows.

Films of the basecoat, then the topcoat, were drawn down onto Laneta Form 5C—Opacity cards and allowed to dry. Gloss measurements were made using a BYK micro-TRI-gloss meter at the 200 and 60° reflectance angles.

The results obtained demonstrated a high level spectral reflectance effect. With Gloss @ 200=473 and Gloss @ 600=444.

What is claimed is:

1. A nail composition set comprising a basecoat composition comprising mirror pigment and a topcoat composition comprising at least one film-forming agent and a solvent component comprising polar solvent.

2. A kit comprising at least one basecoat composition comprising mirror pigment and at least one topcoat composition comprising at least one film-forming agent and a solvent component comprising polar solvent.

3. A method of maintaining mirror properties of a basecoat composition which has been applied to a nail comprising mirror pigment comprising applying a topcoat composition comprising at least one film-forming agent and a solvent component comprising polar solvent to the basecoat composition.

4. The nail composition set of claim 1, wherein the basecoat composition further comprises at least one pearlescent agent, and the weight ratio of pearlescent agent to mirror pigment is from about 20:1 to about 1:1.

5. The kit of claim 2, wherein the basecoat composition further comprises at least one pearlescent agent, and the weight ratio of pearlescent agent to mirror pigment is from about 20:1 to about 1:1.

6. The method of claim 3, wherein the basecoat composition further comprises at least one pearlescent agent, and the weight ratio of pearlescent agent to mirror pigment is from about 20:1 to about 1:1.

7. The nail composition set of claim 1, wherein the solvent component of the topcoat composition consists essentially of polar solvent selected from the group consisting of water, C2-C5 alcohols, and mixtures thereof.

8. The kit of claim 2, wherein the solvent component of the topcoat composition consists essentially of polar solvent selected from the group consisting of water, C2-C5 alcohols, and mixtures thereof.

9. The method of claim 3, wherein the solvent component of the topcoat composition consists essentially of polar solvent selected from the group consisting of water, C2-C5 alcohols, and mixtures thereof.

10. The nail composition set of claim 1, wherein the mirror pigment is an aluminium pigment having (1) a hydrophobic surface treatment and (2) a length to thickness ratio of 10 to 100 and/or a width to thickness ratio of 10 to 100.

11. The kit of claim 2, wherein the mirror pigment is an aluminium pigment having (1) a hydrophobic surface treatment and (2) a length to thickness ratio of 10 to 100 and/or a width to thickness ratio of 10 to 100.

12. The method of claim 3, wherein the mirror pigment is an aluminium pigment having (1) a hydrophobic surface treatment and (2) a length to thickness ratio of 10 to 100 and/or a width to thickness ratio of 10 to 100.

13. The nail composition set of claim 1, wherein the mirror pigment is present in an amount ranging from about 0.075% to about 1% by weight, based on the total weight of the basecoat composition.

14. The kit of claim 2, wherein the mirror pigment is present in an amount ranging from about 0.075% to about 1% by weight, based on the total weight of the basecoat composition.

15. The method of claim 3, wherein the mirror pigment is present in an amount ranging from about 0.075% to about 1% by weight, based on the total weight of the basecoat composition.

16. The nail composition set of claim 4, wherein the pearlescent pigment is present in an amount ranging from about 0.5% to about 1% by weight, based on the total weight of the basecoat composition.

17. The kit of claim 5, wherein the pearlescent pigment is present in an amount ranging from about 0.5% to about 1% by weight, based on the total weight of the basecoat composition.

18. The method of claim 6, wherein the pearlescent pigment is present in an amount ranging from about 0.5% to about 1% by weight, based on the total weight of the basecoat composition.

19. The nail composition set of claim 1, wherein the topcoat composition has haze properties of 700 HU or less.

20. The method of claim 3, wherein the topcoat composition has haze properties of 700 HU or less.
